(19) United States
(12) Patent Application Publication
(10) Pub. No.: US 2017/0055017 A1
(43) Pub. Date: Feb. 23, 2017

(54) Sensed Content Delivery

(71) Applicant: PIKSEL, INC., Wilmington, DE (US)

(72) Inventors: Mark Christie, London (GB); Ralf Wilhelm Tillmann, Mannheim (DE)

(21) Appl. No.: 15/119,097

(22) PCT Filed: Feb. 13, 2015
(86) PCT No.: PCT/EP2015/053144

§ 371 Date: Aug. 15, 2016

(30) Foreign Application Priority Data
Feb. 13, 2014 (GB) 1402536.5

(51) Int. Cl. H04N 21/422; H04N 21/442; A61B 5/00; H04N 21/435; G06F 3/01; A61B 5/16; H04N 21/845; H04N 21/472

(52) U.S. Cl. CPC H04N 21/42201; H04N 21/8456; H04N 21/44218; H04N 21/47202; H04N 21/435; G06F 3/011; A61B 5/165; A61B 5/486; G06F 2203/011

(57) ABSTRACT

Embodiments of a computer implemented method for controlling media content in an environment of a consumer are described, the embodiments having at least one media device for media consumption by the consumer, the method comprising at least one processor configured to provide media content to the media device for consumption by the consumer; receive, from at least one sensor sensed body data of the at least one consumer indicating a state of the consumer; and control the environment or physical state of the consumer in dependence on the sensed body data to modify the indicated state.

### TECHNICAL FIELD

The present invention relates to content delivery. In particular, the present invention relates to methods and systems for content delivery based on audience sensing.

### BACKGROUND

There is an ever increasing amount of media content, provided by for example digital or analogue television channels to consumers or viewers, either broadcast or via a network such as the internet. There is also an ever increasing consumer preference for “on-demand” content which can be selected and viewed by a consumer on an or their appropriate media device, such as a television or flat screen, tablet or laptop or mobile phone, at a time appropriate for the consumer, or if there are many consumers constituting an audience, at a time appropriate for the audience.

Navigating through the ever increasing amount of media content, whether on-demand or not, can still however be time consuming. Furthermore, traditional media content may not fit the ever-changing needs and desires of the consumer or audience, even if such media content was initially selected by the consumer or audience. Such challenges are increased when one considers the changing and perhaps differing desires of more than one consumer during the consumption of media content at the same time.

Furthermore, some consumers may have a medical condition or conditions that may impair their emotional, physical or mental abilities, to one extent or another depending on the condition. In some circumstances, such consumers may inhabit an environment with systems that monitor, diagnose and administer prescribed medication in accordance with the condition.

Matching the desires and wants of such consumers whilst consuming media content in a safe way with respect to their pre-existing condition(s) is technically challenging.

### SUMMARY

According to one aspect there is provided a computer implemented method for controlling media content in an environment of a consumer, the environment also having at least one media device for media consumption by the consumer, the method comprising at least one processor configured to provide media content to the media device for consumption by the consumer; receive, from at least one sensor sensed body data of the at least one consumer indicating a state of the consumer; and control the environment or physical state of the consumer in dependence on the sensed body data to modify the indicated state.

Hence, the environment of consumer is controlled by altering the physical state or environment itself to modify the detected sensed body state.

An embodiment may comprise altering or providing a prescribed medication.

An embodiment may comprise altering the media content being provided to the consumer. For example, if the consumer is becoming stressed or agitated, then the media content may be altered to content more soothing or less exciting, with monitoring of the body state informing the progress of such.

Alternatively, or in addition, the media content may be stopped in other embodiments.

The sensed body data may be biometric data indicating the mood or emotional state of the at least one consumer.

The body state may be a stressed state.

The body state may be an anxious or elevated state.

The controlling may be based on thresholds for certain states, for each particular consumer.

In some embodiments, the biometric data may comprise data indicative of the mood or emotional state of the consumer. For example, the biometric data may comprise one of facial measurement characteristics, body measurement characteristics, heartbeat, pulse, temperature, skin resistance, blood sugar levels, blood pressure, oxygen saturation levels, blink rate, voice signals, voice level and tone, iris pattern data.

For example, indicators of mood may comprise body sense data measurements indicating smiling, frowning, or crying. Crossed legs or arms may indicate a negative (not open) feeling or mood, and raised voice, or heartbeat and skin resistance, blood sugar levels, and/or blood pressure, pulse, voice tone, may indicate excitement, or stress levels or anxiety.

A feedback loop between the biometric sensor(s) and the environment and the control system or module may provide a method or system embodiment that may intervene to modify the environment of the consumer so as to modify the sensed state of the consumer. This may apply to moods or states that are determined to be perhaps harmful or dangerous for the consumer depending on the medical condition the consumer has.

The environment may contain medical devices to aid controlling of the state.

In another embodiment, the sensed body data may be aggregated over time.

In another embodiment, the sensed body data may be received from more than one sensor.

In yet another embodiment, the sensed body data may be received from more than one sensor at one time, and/or aggregated over time.

In another embodiment, the sensed body data may be received from more than one consumer.

In another embodiment, the media content may be arranged in multiple fragments in the data store.

In an embodiment, the multiple fragments may be linked multidimensionally based at least in part on sensed body data, and may further be optionally linked by scene decision metadata.

In an embodiment, the selection of media content may further comprise computing a vector based on the sensed body data and scene decision metadata, the output comprising a plurality of fragments for output.

In another embodiment, the control of the output of the media content may comprise stopping the output, and said control of stopping may be optionally dependent on a pre-set, or consumer set body sensed threshold.

According to a second aspect, there is provided a system for controlling media content in an environment of a consumer, the environment also having at least one media device for media consumption by the consumer and at least one sensor for sensing body data of the consumer and a receiving means for receiving the data of said at least one sensor, the system further comprising at least one processor configured to provide media content to the media device for consumption by the consumer; receive via receiving means, from the at least one sensor in the environment, sensed body data of the at least one consumer indicating a state of the consumer; and control the environment or physical state of the consumer in dependence on the sensed body data to modify the indicated state.

An embodiment may comprise altering or providing a prescribed medication.

An embodiment may comprise altering the media content being provided to the consumer. For example, if the consumer is becoming stressed or agitated, then the media content may be altered to content more soothing or less exciting, with monitoring of the body state informing the progress of such.

Alternatively, or in addition, the media content may be stopped in other embodiments.

The sensed body data may be biometric data indicating the mood or emotional state of the at least one consumer.

The body state may be a stressed state.

The body state may be an anxious or elevated state.

The controlling may be based on thresholds for certain states, for each particular consumer.

In an embodiment of this aspect, the at least one sensor for sensing body data of the at least one consumer may be adapted to sense biometric data.

In other embodiments, the at least one sensor adapted to sense biometric data may comprise means for sensing at least one of facial measurement characteristics, body measurement characteristics, heartbeat, pulse, temperature, skin resistance, blood sugar levels, blood pressure, oxygen saturation levels, blink rate, voice signals, voice level and tone, iris pattern data.

A feedback loop between the biometric sensor(s) and the environment and the control system or module may provide a method or system embodiment that may intervene to modify the environment of the consumer so as to modify the sensed state of the consumer. This may apply to moods or states that are determined to be perhaps harmful or dangerous for the consumer depending on the medical condition the consumer has.

The environment may contain medical devices to aid controlling of the state.

In another embodiment, the processor may be configured to aggregate the sensed biometric data over time.

In another embodiment, the system may comprise more than one sensor and the sensed body data may be received from said more than one sensor.

In an embodiment, the sensed body data may be received from more than one consumer.

In yet another embodiment, the data store may store media content in multiple fragments, and optionally the multiple fragments may be linked multidimensionally based at least in part on sensed body data and/or by scene decision metadata.

In another embodiment, the processor may be configured to select media content for output by computing a vector based on the sensed body data and scene decision metadata.

In yet another embodiment of this aspect there is provided a scene insertion engine for controlling the insertion and synchronising of said output to said at least one media device.

### BRIEF DESCRIPTION OF THE DRAWINGS

For a better understanding of the present disclosure and to show how the same may be put into effect, reference will now be made, by way of example only, to the following drawings in which:

FIG. 1 illustrates an environment of a viewing system according to an embodiment;

FIG. 2 is a flow chart for a process of delivering content to consumer(s) according to an embodiment; and

FIG. 3 shows a modular schematic view of a system according to another embodiment; and

FIG. 4 shows a process according to an embodiment.

### DETAILED DESCRIPTION

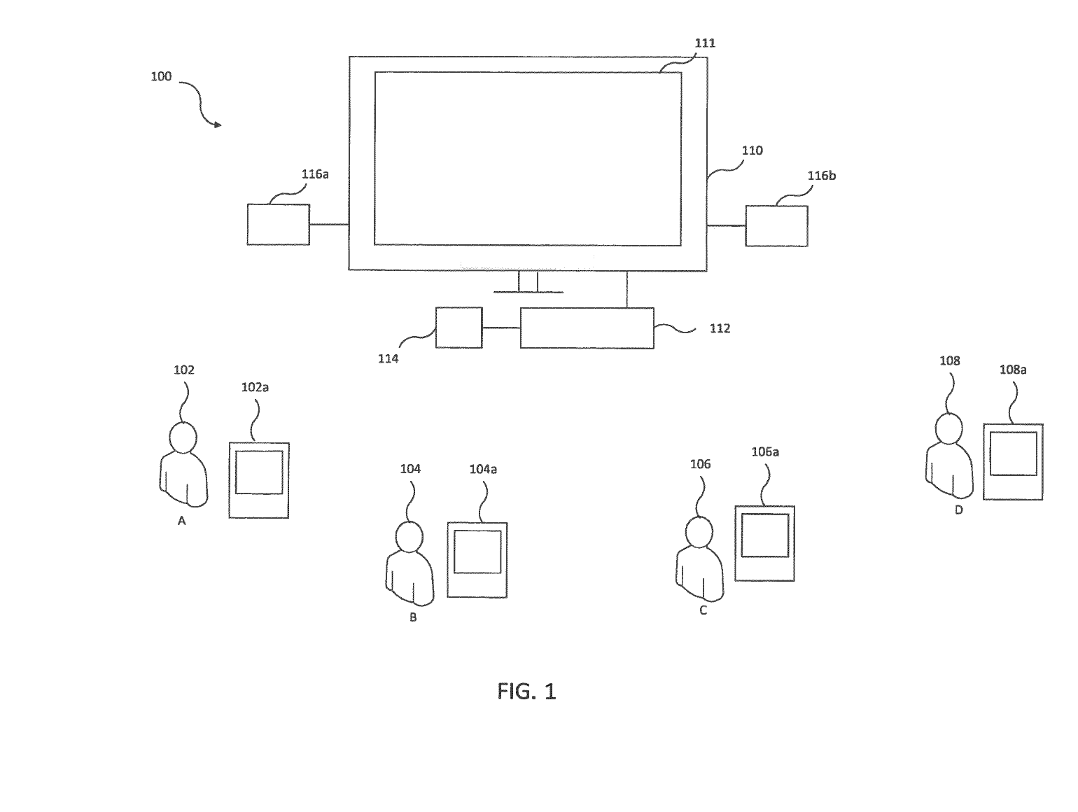

FIG. 1 illustrates an environment having a system 100 comprising at least one media device 110 such as a television with a screen or display 111. The at least one media device 110 is coupled to a video playback device 112 which is configured to display media content on the screen 111 of the at least one media device 110. The video playback device 112 may comprise for example a set top box, a digital media player, or a video disc player etc. The video playback device 112 is coupled to receiver means 114. The receiver means 114 is configured to receive sensed data of consumers in the environment of the system 100. The sensed data is for use by the video playback device 112 as will be described later. Whilst FIG. 1 shows the video playback device 112 and the receiver means 114 as external to the viewing devices, it will be readily appreciated by those skilled in the art that the functionality of one or more of the video playback device 112 and the receiver means 114 may be incorporated into the viewing device 110.

The at least one media device 110 may be coupled to one or more media output devices such as speakers 116a, 116b for outputting audio signals. The one or more media output devices 116a, 116b may be external to the at least one media device 110 and coupled to the at least one media device 110 using a suitable interface (as shown in FIG. 1). The interface may be wired or wireless. Alternatively, the one or more speakers 116a, 116b may be integrated into the at least one media device 110.

The at least one media device(s) described above are exemplary only. The at least one media device(s) may comprise other synaptic or for example haptic output devices. For example a reconfigurable braille reading device may comprise, or be incorporated in said at least one media device capable of outputting content (in whatever form) to the consumer(s).

As shown in FIG. 1, a plurality of consumers may be present in the environment of the system 100 and want to watch or consume content delivered using the at least one media device 110. FIG. 1 shows a first consumer 102 (A), a second consumer 104 (B), a third consumer 106 (C), and a fourth consumer 108 (D). Whilst four consumers are shown as being present in the environment of the system 100, this number of consumers is merely an example. In some embodiments there may be only one consumer. In other embodiments there may be many consumers, such as in environments comprising theatres or cinemas for example.

Those skilled in the art will appreciate that, in some embodiments, there may be more than one media device. For example, each consumer, whilst in the same environment, may however be consuming media content on his or her own device capable of media output. This may for example, be the consumer's mobile phone, or “phablet”, or tablet PC, or laptop, or could in some examples comprise a handheld gaming device and/or an audio device such as a digital music player.

The at least one media device may also comprise device systems including haptic and/or synaptic devices. Other examples comprise head mounted or worn projection displays (“glasses”) for virtual reality or augmented reality applications, or any combination of these examples.

One or more of the plurality of consumers may be associated with a sensing device configured to sense a mood or emotional state of the user. For example consumer A 102 may be associated with a first sensing device 102a, consumer B 104 may be associated with a second sensing device 104a, consumer C 106 may be associated with a third sensing device 106c, and consumer D 108 may be associated with a fourth sensing device 108a.

The sensor or sensing devices may be, for example, stand-alone biometric sensor devices, or the sensors may be integrated into a user's mobile phone, personal digital assistant (“PDA”), a personal computer (“PC”), a tablet computer, a gaming device or other example media devices as described above, or may be sensors able to communicate with applications or “apps” for example running on such devices. Such sensors and/or devices are able to communicate with the system 100 by for example the receiver means using well known wireless or wired or optical interfaces and network means.

By way of example only, and without limitation, the sensing device(s) may be adapted to sense at least one or more one of facial measurement characteristics, body measurement characteristics, heartbeat, pulse, temperature, skin resistance, blood sugar levels, blood pressure, oxygen saturation levels, blink rate, voice signals, voice level and tone, iris pattern data.

Biometric information indicating or associated with the mood or emotional state of the consumer may be sensed by the sensing devices 102a, 104b, 106c, 108d associated with their respective consumers.

### Sensors and Mood

The inventors have realised that feedback in the form of emotional mood or state of a consumer or consumer could be used in real time to adapt or alter the media content being consumed, so as to optimise the experience for the consuming consumer. For example, a consumer may select a romantic movie to view if in that mood, and then subsequently during consumption, the system may detect that the consumer is not enjoying the movie. In such a scenario, the system may then select alternative scene branches for the media content based on the sensed biometric data. In the preceding example, for instance, the system may select scenes representing a happier narrative arc or ending, with feedback from the sensors further being utilised to select further media fragments comprising appropriately linked scenes.

Another example scenario may comprise a film or movie in the “horror” genre. Biometric feedback may influence the control of scene selection if it is detected that the consumer(s) are “too frightened” or “upset”. For example, a combination of pulse rate, blood pressure and/or voice tone data may indicate such an emotional state. In such a scenario alternative scenes may be selected for outputting, for example scenes in which a certain character “escapes death” or perhaps scenes which are less “gory” or “blood spattered” may be controlled for selection and output.

Alternatively, in another embodiment, the output of the media content could be controlled to be stopped overall, if the system determines so on a pre-set, or consumer set threshold.

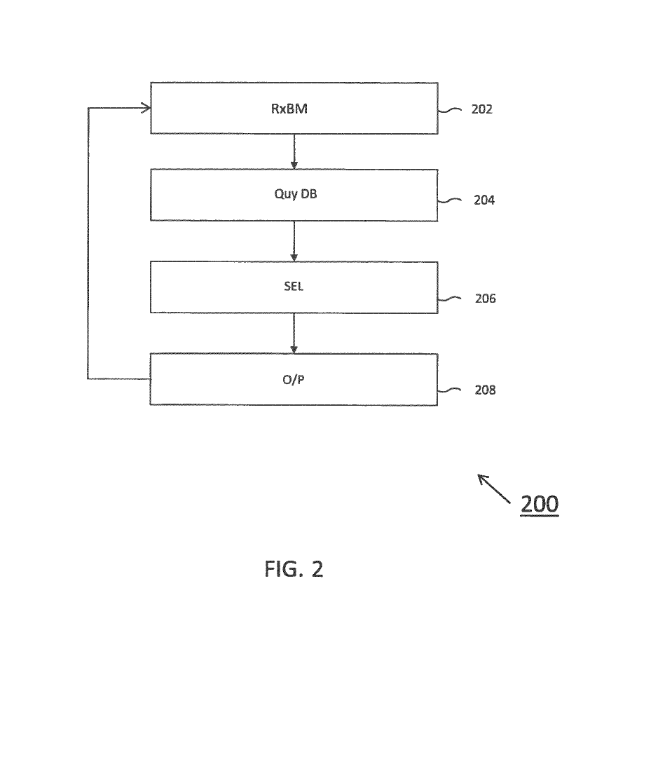

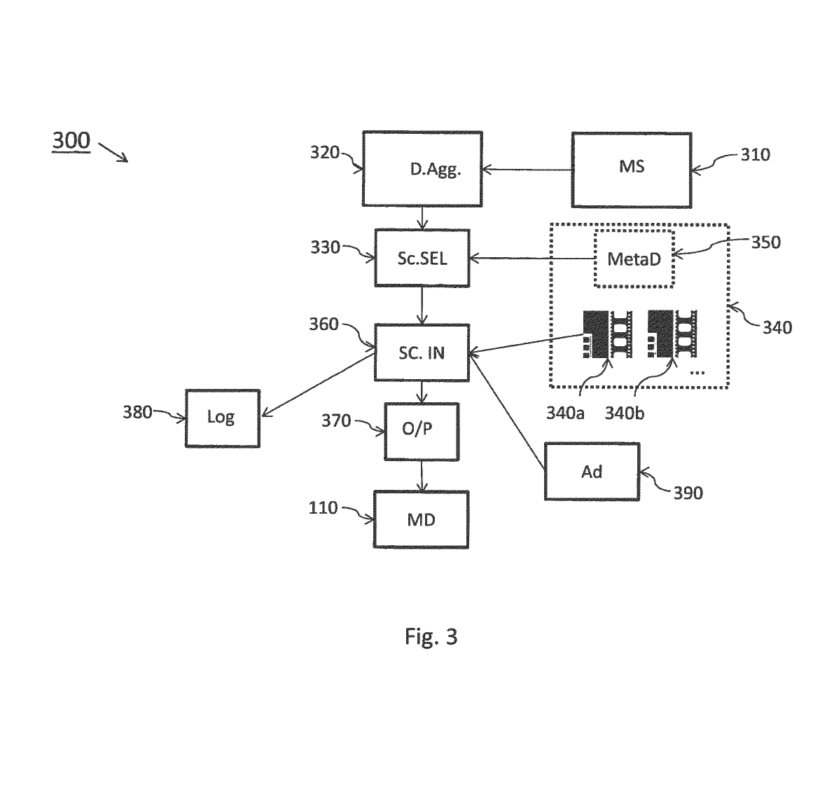

This will now be elaborated on with reference to FIG. 2 which illustrates an example method 200 performed by the system of FIG. 1 (or FIG. 3).

In this embodiment, the sensors 102a, 104b, 106c, 108d provide body sensed data indicating the mood or emotional state of the respective consumer at step 202.

The body sensed data may comprise at least one or more of facial measurement characteristics, body measurement characteristics, heartbeat, pulse, temperature, skin resistance, blood sugar levels, blood pressure, oxygen saturation levels, blink rate, voice signals, voice level and tone, iris pattern data.

By way of example, data indicating a scared mood may be represented by combining biometric data from heart rate (increased), blood pressure (increased), voice level (quiet or whimpering) and an increased blink rate. Such data may be compared by the at least one processor of the system against a threshold to indicate the mood.

Those skilled in the art will recognise that many combinations, depending on the available sensors may be constructed to determine thresholds.

In another embodiment, data collected from the same consumer may, over time, be used as a baseline to determine thresholds for that consumer, with feedback from the consumer aiding fine tuning of such thresholds.

In another embodiment, the thresholds may be set such that output is actually controlled to stop. i.e. the biometric data passes a threshold associated with stopping the content (which may for example indicate that the consumer was not enjoying the content at all, or that the consumer was for example too upset or scared to carry on consuming the content.)

The threshold may have parental input for children, to help control the media content they consumer.

This data (RxBM) is received 114 by the system 100.

The system processor then queries a datastore or memory 340 at step 204 (Quy DB), the datastore or memory storing media content.

The process then flows to step 206 (SEL) at which appropriate media content or fragments or scenes based on the sensed body data are selected, and such selected media content is then controlled for output at step 208 (O/P).

The process then returns to step 202 to constantly receive body sensed data and further select and control appropriate media content in dependence on the monitored and sensed body data.

Hence, the control of the media content selected for output to the at least one consumer may be substantially continuously based on the sensed body data indicating the current mood or state of the at least one consumer.

Note that different media content may be selected for output to different consumers 102 (A), 102 (B), 102 (C), 102 (D) if they are consuming such content on their own media devices wither in the vicinity of the system 100 or if they are a virtual crowd connected via for example the internet to the system 100. The media output may not only comprise different versions of the same overall content (e.g. language versions), but in some embodiments may comprise differing endings or story or narrative arcs of the same overall content in dependence on each consumers sensed body data.

In some embodiments the sensed body data for one consumer 102 (A) from more than one sensor may be aggregated to provide an overall indicator to the system 100 of the current mood or changing state of the consumer.

In some embodiments the sensed body data for more than one consumer 102 (A), 102 (B), 102 (C), 102 (D) from more than one sensor 102a, 104a, 106a, 108a, may be aggregated to provide an overall indicator to the system 100 of the current mood or changing state of the multiple consumers or audience.

In other embodiments each consumer may have more than one associated sensor 102a, 104a, 106a, 108a which provide body sensed data at substantially the same time to the system 100.

In an embodiment. a feedback loop between the biometric sensor(s), a medical device and the control system or module therefore provide a system that may intervene to control the output (or even stop the output) if the consumer is sensed to be in a mood or state perhaps harmful or dangerous depending on the medical condition the consumer has.

Furthermore, the medical device may also administer appropriate medicine dosages (e.g. insulin if diabetic) depending on the sensed body data and condition.

States may include, in some embodiments, being anxious, stressed, excited, over excited, wheezing (hard of breath), and so on.

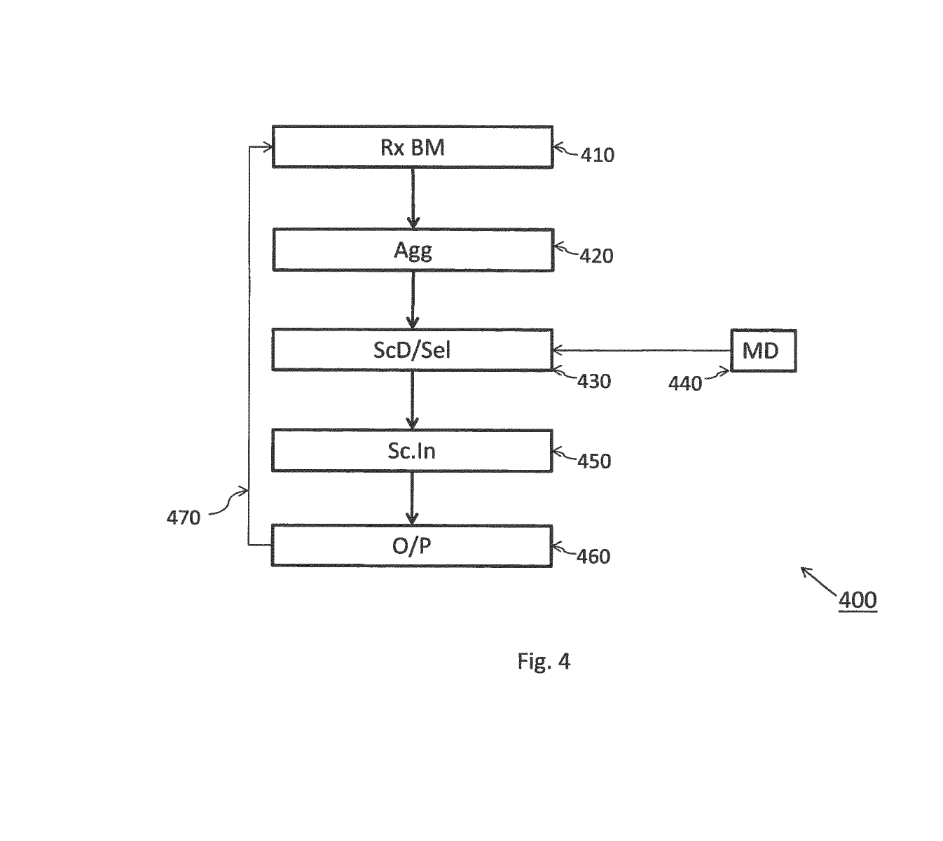

Another embodiment now be described with reference to FIGS. 3 and 4.

FIG. 3 illustrates modular functional blocks of an environment having system embodiment 300. The system comprises a monitoring system 310 which receives audience or consumer sensed body data. This data is communicated to a data aggregation module 320 which may sort and average the data over time, or per consumer, or a combination of both for example. The aggregated data is passed from the data aggregation module 320 to a scene selection module 330.

The scene selection module is connected to a datastore 340 suitable for storing media content.

In FIG. 3, the media content is stored as a selection of multiple media fragments 340a, 340b comprising scenes of a movie or film for example. The media fragments may be linked multidimensionally. For example, a film may have a beginning arc comprising a multiple number of media fragments linked temporally one after the other. The film may also then have several different “middle” arcs or portions or narrative tracks, and similarly several different ending arcs or portions or narrative tracks or pathways. One may visualise such data as multi-dimensionally arranged since the fragments or story lines are non-linear.

In this embodiment there is also provided metadata 350 linking the fragments 340a, 340b with sensed body data characteristics.

The metadata may be linked to the varying arcs or pathways through the media content, characterising such arcs therein. For example, body data indicating an unhappy mood may be linked to arcs that display a happy ending for the movie, and so on. Those skilled in the art will appreciate that many variations on the linking depending on the media content and sensed body data may be utilised.

The system also comprises a scene insertion engine or module 360 for inserting the selected media fragments in synchronised order to an output module 370 connected to at least one media device 110 for presenting the media content to the consumer or consumers for consumption.

The system may also optionally, in some embodiments, have a logging module 380 connected to the scene insertion engine 360. The logging module 380 may log system performance to aid optimisation of said performance against predetermined criteria such as for example, NV sync error, network lag and so on as recognised by those skilled in the art.

The system may also optionally, in some embodiments, have an advertising module 390 connected to the scene insertion engine 360. The advertising module may then offer additional media content in the form of advertisements to the scene insertion engine 360. Such advertisements may be linked to the sensed body data to help target the adverts based on the mood of the consumers or users.

FIG. 4 illustrates an embodiment process flow 400 as may be carried out by the system embodiment of FIG. 3.

At step 410 the monitoring system 310 receives audience or consumer sensed body data (RxBM) and communicates this at step 420 (Agg) to the data aggregation module 320 which may sort and average the data over time, or per consumer, or a combination of both for example. The aggregated data is passed from the data aggregation module 320 to a scene selection module 330 which selects at step 430 scenes as described above with reference to FIG. 3 based on the stored 340 media fragments and metadata 440 provided as input to the scene selection module 330.

Flow then passes to the scene insertion engine 360 at step 450 which synchronises and inserts the selected media content to control output at step 460 to the at least one consumer's (or consumers') media devices.

Flow then passes 470 back to the monitoring and receiving (RxBM) of body sensed data at step 410.

Hence, media content is controlled for selection and output substantially in real time based on sensed body data indicating the mood or emotional state of the consumer, or consumers, or audience. Furthermore, the constant monitoring of the state enables further selection and update control in order to try to match the mood of the consumer(s) as his or her mood changes during the consumption of the media content.

In the above a content delivery system for controlling media content provided by at least one media device for media consumption by at least one consumer is described. In an embodiment the system comprises at least one sensor for sensing body data of the at least one consumer, a receiver for receiving sensed body data from the sensor, a data store storing media content for selection, at least one media device for outputting said selected content to said at least one consumer, and at least one processor configured to query said data store and control the selection of said media content based at least in part on the sensed body data, and control the output of said selected media content to said at least one media device. The sensed body data may indicate the mood or emotional state of the consumer.

It will be appreciated that the above embodiments have been described only by way of example, and other variants or applications may be apparent to a person skilled in the art given the disclosure herein.

The present invention is not limited by the described examples but only by the appendant claims.

1. A computer implemented method for controlling media content in an environment of a consumer, the environment also having at least one media device for media consumption by the consumer, the method comprising at least one processor configured to:
provide media content to the media device for consumption by the consumer;
receive, from at least one sensor sensed body data of the at least one consumer indicating a state of the consumer; and
control the environment or physical state of the consumer in dependence on the sensed body data to modify the indicated state.

2. A method according to claim 1, further comprising the step of stopping said provided media content.

3. A method according to claim 1, further comprising the step of altering said provided media content.

4. A method according to claim 1, wherein the state of the consumer is a stress state.

5. A method according to claim 4, wherein the state is reduced or increased.

6. A system for controlling media content in an environment of a consumer, the environment also having at least one media device for media consumption by the consumer and at least one sensor for sensing body data of the consumer and a receiving means for receiving the data of said at least one sensor, the system further comprising at least one processor configured to:
provide media content to the media device for consumption by the consumer;
receive via receiving means, from the at least one sensor in the environment, sensed body data of the at least one consumer indicating a state of the consumer; and
control the environment or physical state of the consumer in dependence on the sensed body data to modify the indicated state.

7. A system according to claim 6, wherein said processor further controls by stopping said provided media content.

8. A system according to claim 6, wherein said processor further controls by altering said provided media content.

9. A system according to claim 6, wherein the state of the consumer is a stress state.

10. A method according to claim 9, wherein the state is reduced or increased.
